(19) United States
(12) Patent Application Publication
(10) Pub. No.: US 2024/0240262 A1
(43) Pub. Date: Jul. 18, 2024

(54) VIABLE CELL DETECTION AND PROTOCOL IMPLEMENTING THE SAME

(71) Applicant: Trace-Ability, Inc., Van Nuys, CA (US)

(72) Inventors: Eugenia Khorosheva, South Pasadena, CA (US); Paolo Benigni, Los Angeles, CA (US); Arkadij M. Elizarov, Woodland Hills, CA (US)

(21) Appl. No.: 18/395,034

(22) Filed: Dec. 22, 2023

Related U.S. Application Data
(60) Provisional application No. 63/434,641, filed on Dec. 22, 2022.

(51) Int. Cl. C12Q 1/6888

(52) U.S. Cl. CPC C12Q 1/6888

(57) ABSTRACT

A viable cell testing method, such as a sterility test, comprising the steps of obtaining a sample; conditioning the sample by maximizing viability of at least one RNA based biomarker, wherein the RNA based biomarker is preferably universal, abundant and short-lived; treating the sample to facilitate reagent contacting the RNA based biomarker; amplifying at least one nucleic acid sequence of the RNA based biomarker using amplification reagents; interacting with the amplified sequences of the RNA based biomarker to produce a readout signal; and detecting the readout signal and determining the presence of viable cells of the sample. A pharmaceutical protocol for a short-lived pharmaceutical implements the viable cell testing method, administering the pharmaceutical to the patient following safety confirmation and within three hours of obtaining the sample from the patient.

### CROSS-REFERENCE TO RELATED APPLICATIONS

This application is a continuation of and claims the benefit of Provisional Patent Application No. 63/434,641 filed Dec. 22, 2022, the entire contents of which are incorporated by reference in their entirety.

### STATEMENT REGARDING FEDERALLY SPONSORED RESEARCH

This invention was developed, in part, with government support under grant number R43FD006914 awarded by the U.S. Food and Drug Administration. The government has certain rights in the invention.

### BACKGROUND OF THE INVENTION

### Field of the Invention

This invention generally relates to detection of viable cells and applications of microbial detection, such as sterility testing, and to pharmaceutical, medical, food and beverage manufacture and industrial protocols implementing the same, and to kits and systems for performing the same.

### Background Information

Culture-based microbial detection and protocols implementing the same are currently used in the industry for sterility testing. Culture-based techniques are time consuming, requiring multiple days and requiring labor and infrastructure. Culturing techniques target culturable viable cells, including active microorganisms, dormant cells, and spores, but do not include dead cells, i.e., cells which are incapable of replication. Viable cells typically refer to living cells, such as cells performing protein synthesis and having intact cell membranes.

Sterility is the absence of any viable microorganisms. As long as there is at least one living cell, or a single spore that can develop into a living cell, in the sample, the sample is not sterile. A given sample can be considered sterile when it is shown through accepted protocols that it does not contain even a single viable cell or spore.

Sterilization processes are applied to products in a number of industries, including food and beverage manufacture, but it is mainly in the pharmaceutical and medical sectors where the sampling of sterile products for testing remains an important routine task for microbiologists.

Sterility assurance is critical in the manufacture of many drugs and other medical products and is closely regulated worldwide. Products carrying a claim of sterility typically require some form of sterility test to be carried out before release in order to verify that claim. Ideally, a sterilization process (e.g. heat, or ionizing radiation) would be applied to the product in the final container at the end of the manufacturing process. However, some products contain heat-, or radiation-sensitive components, which cannot be terminally sterilized in pack. These products are often sterilized by filtration, then filled aseptically.

A sterility test in the pharmaceutical and medical product space is essentially a test which assesses whether a sterilized pharmaceutical or medical product is free from contaminating microorganisms and is conventionally performed by incubation of either the whole or a part of that product with a nutrient medium.

The sterility test does have an important application in monitoring the microbiological quality of filter-sterilized, aseptically filled products and does offer a final check on terminally sterilized articles. The sterility test remains an important tool for pharmaceutical microbiology laboratories to determine conformance where there is a claim that a product is sterile. Pharmaceutical regulations, such as the United States Pharmacopeia, the European Pharmacopocia, or Japanese Pharmacopeia, prescribe procedures that must be followed. They provide the details of the sample sizes and conditions to be adopted in particular cases. While sterility testing may be required or recommended by regulations governing the pharmaceutical industry, it plays a relatively minor role in sterility assurance. By far the greatest contribution to sterility comes from the validation and control of the sterilization process, and/or of aseptic processing procedures.

Today's conventional, culture-based, sterility testing presents several significant limitations in pharmaceutical and medical product quality control. Most notably, it is time consuming, typically requiring 14 days to execute for traditional methods. It also requires labor and infrastructure. Clearly, this is a delay that is becoming less and less acceptable in a modern manufacturing operation.

The culturing-based techniques are unsuitable for pharmaceutical or medical products with a short shelf-life and/or those intended for immediate use. These pharmaceutical products include, for example, Positron Emission Tomography (PET) tracers, CAR-T cells and new cell therapy products, platelet transfusion products, gene therapies, and tissue engineered products. These products are often administered to patients before a standard sterility test may be completed.

Initiatives such as ‘Process Analytical Technology’ (PAT) and parametric product release are challenging the need for sterility tests to be completed before product can be released. Both the FDA and the EMEA are encouraging the adoption of new analytical technologies to help ensure final product quality. In the USA the FDA Center for Biologics Evaluation and Research (CBER) is proposing radical changes to the sterility test requirements for biological products, though not so far for all pharmaceuticals, promoting the use of rapid microbiological methods (RMM) as alternatives to the traditional sterility test methods. Such changes in the regulatory climate are creating renewed interest in RMM for sterility testing and several technologies are already available commercially.

ATP-bioluminescence Adenosine triphosphate (ATP) bioluminescence is an established rapid method utilizing a specific substrate and enzyme combination, luciferin/luciferase, to break down microbial ATP from growing cells and produce visible light, which can be measured using a luminometer. Several commercial systems have been developed for a range of pharmaceutical test applications, including sterility testing, especially for filterable samples where non-microbial ATP in the sample is less of a concern. The test time can be reduced considerably because detection of microbial growth in culture media is accomplished by ATP-bioluminescence, rather than by visible turbidity. Typically, results equivalent to those of compendial tests are available within about 7 days.

The Milliflex® Rapid Microbiology Detection and Enumeration system from Millipore also uses ATP-bioluminescence to detect microbial cells and is designed specifically for monitoring microbial contamination in filterable samples. It is automated, employing image analysis technology to detect micro-colonies growing directly on the surface of a membrane filter after the addition of bioluminescence reagents. The system is designed to be quantitative, but a method has been developed and validated to use it for a rapid sterility test with an incubation time of five days.

Colorimetric growth detection methods rely on a color change being produced in a growth medium as a result of microbial metabolism during growth, often as a result of CO2 production. One example of a commercial colorimetric assay system, which can be used for sterility testing is the BacT/ALERT® 3D Dual-T Microbial Detection System from bioMerieux. The system is automated and employs sensitive color detection and analysis technology to produce a result in as little as three days. It can detect both acrobic and anaerobic bacteria, as well as yeasts and molds.

Cytometry systems have been proposed which do not rely on microbial growth to detect contamination, but instead uses cell labelling techniques to detect viable microorganisms. This approach has the potential to detect a wide range of organisms, including yeasts and molds, within minutes. Commercial systems utilize combined fluorescent cell labelling and flow cytometry or solid phase cytometry to detect viable microbial cells. Typically, the cells are labelled using a fluorescent dye or a non-fluorescent substrate, which is converted to a fluorochrome in viable cells. Detection of the labelled cells occurs by laser scanning in either a flow cell (flow cytometry), or on a solid phase platform such as a membrane filter (solid phase cytometry). AES Chemunex has developed solid phase cytometry detection systems. The company's Scan®RDI (also known as ChemScan RDI) system is alleged to be capable of detecting 1 CFU per sample and has been evaluated as a possible RMM for sterility testing. The technology has been developed for the Stereal®-T sterility testing system.

Viability PCR (vPCR) approach assesses cell membrane integrity. Propidium monoazide (PMA) covalently binds DNA in cells with compromised cell membranes and inhibits amplification by PCR upon photoactivation. In this approach, false-positive results are expected for inactivated cells that retain intact cell membranes. False-negative results are possible for spores and for competent cells. Moreover, DNA is not an abundant enough molecule to allow detecting cells at low concentration (starting from 1 cfu/mL). (Nocker & Camper, 2009), (Emerson et al., 2017).

There is no sterility test and other rapid methods for selective detection of living microorganisms available on market. The published methods are developed for microbiome studies, use with ribosomal rRNA sequencing (such as 16S RNA). Other methods are developed for pathogen detection in samples, where a mere pathogen presence (without needs to distinguish alive or dead) in a clinical sample is evidence of an infection.

The above is a quick sampling of RMM technologies. There remains a need in the industry for rapid and sensitive detection of living prokaryotic and eukaryotic cells at low concentration (starting from 1-5 CFU/mL) and without a culture step (where cellular division takes place typically taking from a few hours to 14 days). Multiple RRM methods were developed for detecting microorganisms for different purposes, but there has been no developed approach which efficiently effectively and consistently yields ultra-rapid detection of living microorganisms at low concentration (1-100 cells/mL), universally, with the goal of only detecting living cells. As tests under seven days are sometimes deemed rapid in the art, thus in the present application the phrase “ultra-rapid”, as associated with viable cell detection or sterility testing, will reference processes yielding results under 2 hours, obviously with no culturing process needed. Solving this ultra-rapid processing need would allow for engineering breakthrough technologies applicable in many fields of medicine and science, and in industries long waiting for solutions for ultra-rapid sterility testing. It would also allow ultra-rapid bioburden analysis in pharmaceutical industry and allow for quality solutions in food and water safety control, and in diagnostics and detection.

### SUMMARY

The various embodiments and examples of the present invention as presented herein are understood to be illustrative of the present invention and not restrictive thereof and are non-limiting with respect to the scope of the invention.

One embodiment of the present description provides a viable cell detection method comprising the steps of: obtaining a sample for detection; conditioning the sample for detection by facilitating an abundance of at least one universal, short-lived RNA based biomarker; performing a reaction to generate a readout signal from at least one nucleic acid sequence of the RNA based biomarker; detecting the readout signal which is indicative of the presence or absence of at least one viable cell within the sample; and analyzing the readout signal and reporting whether the method detects viable cells in the analysis sample.

According to the method, an analysis sample is provided which can include viable cells to be detected, non-viable cells, a combination of viable cells to be detected and non-viable cells, or no cells. A reaction is performed on the analysis sample to detect the abundant short-lived RNA molecules from viable cells in the analysis sample, where at least one nucleic acid sequence of the RNA based biomarker is amplified using amplification reagents. To detect the abundant short-lived RNA molecules, data on the reaction may be obtained and analyzed. Analysis of the data determine one of two outcomes (1) the presence of viable cells in the analysis sample, with or without non-viable cells included in the analysis sample; indicated by detecting the presence of short-lived RNA molecules; or (2) the absence of viable cells or no cells in the analysis sample. When reaction products, e.g., amplification products, are directly or indirectly detected in a threshold amount, the presence of viable cells in the analysis sample is indicated.

Another embodiment of the present description provides a sterility testing method for a pharmaceutical product comprising the steps of: obtaining a sample of the pharmaceutical product for testing; conditioning the sample by facilitating an abundance of at least one universal RNA based biomarker; amplifying at least one nucleic acid sequence of the RNA based biomarker using amplification reagents; detecting a readout signal which is indicative of the presence or absence of at least one viable cell within the sample; and determining the sterility of the sample and of the pharmaceutical product based upon the detected readout signal.

Another embodiment of the present description provides a pharmaceutical protocol for administering a pharmaceutical to a patient comprising the steps of: obtaining a sample of a pharmaceutical; conditioning the sample for testing by facilitating an abundance of at least one RNA based biomarker; amplifying at least one nucleic acid sequence of the RNA based biomarker using amplification reagents; detecting a readout signal which is indicative of the presence or absence of at least one viable cell within the sample; and administering the pharmaceutical to the patient following the determination of the absence of at least one viable cell within the sample. In some embodiments the pharmaceutical is administered to the patient within twelve hours of obtaining the sample from the pharmaceutical. Another embodiment of the present description provides a kit for viable cell detection in an analysis sample. The kit includes one or more conditioning reagents that facilitate conditions in the analysis sample such that the viable cells present in the analysis sample possess abundant short-lived RNA molecules. The kit includes one or more RNA access components for providing access to a short-lived RNA biomarker, present in viable cells in the analysis sample, the components comprising: i) a lysis reagent to access the short-lived RNA molecules in the analysis sample; ii) a release reagent to access the short-lived RNA molecules in the analysis sample; iii) a capture agent to access the short-lived RNA molecules in the analysis sample; and a physical agent to access the short-lived RNA molecules in the analysis sample. One or more detection agents for detecting the presence of the short-lived RNA biomarker, where the detection agent is directly or indirectly detectable to indicate the presence of short-lived RNA which is found in viable cells and not in non-viable cells.

The kit may include an imaging agent for detecting the presence of the short-lived RNA biomarker according to another embodiment of the description. Also included in the kit may be additional components and devices including RNA access reagent(s), one or more positive or negative control reagents, and reagents and materials for nucleic acid amplification. In some embodiments, the kit may include stains or one or more oligonucleotide probes for RT-PCR or

RT-LAMP which target the short-lived RNA biomarker as a detection reagent. In some embodiments, the biomarker is non-coding short lived RNase P RNA subunit (RPR) and the probe is specific to either conserved regions (CR1, CR2, CR3, CR4, CR5) in the RPR, or to variable regions between CR1 and CR5.

Another embodiment of the present invention provides a kit for determining the presence of viable cells in an analysis sample, the kit comprising: RNA access components for providing access to, short-lived RNA molecules present in viable cells in an analysis sample, the components comprising: i) one or more conditioning reagents that provide conditions in the analysis sample such that the viable cells present in the analysis sample possess abundant short-lived RNA molecules; and ii) a lysis reagent, release reagent, poration consumable, or capture agent to access the short-lived RNA molecules in the analysis sample; and one or more RT-PCR or RT-LAMP primer sets which target a short-lived RNA biomarker, where the biomarker is RNase P RNA (RPR), where primer sets anneal to a complete or partial sequence of the CR1, CR2, CR3, CR4, and CR5 conserved regions, and where the RT-PCR or RT-LAMP primer sets are directly or indirectly detectable to indicate the presence of the biomarker RNA which is found in viable cells and not in non-viable cells.

Also described herein are systems for viable cell detection in a sample which may incorporate the kit as described herein as well as other components including one or more reaction vessels, an area for modifying the temperature of one or more reaction vessels, an area for manipulating the cell to allow access to cell contents, and an area for biomarker detection.

In some embodiments, the methods, protocols, and kits and systems, as described herein, are for ultra-rapid viable cell detection.

### BRIEF DESCRIPTION OF THE FIGURES

These and other features, aspects and advantages of the present invention will become better understood from the following description, appended claims, and accompanying figures in which like reference numerals represent like elements throughout, where:

FIG. 1 is schematic flow chart of a viable cell detection method according to one embodiment of the present invention.

FIG. 2 is a schematic flow chart of a pharmaceutical protocol for administering a pharmaceutical to a patient according to one embodiment of the present invention.

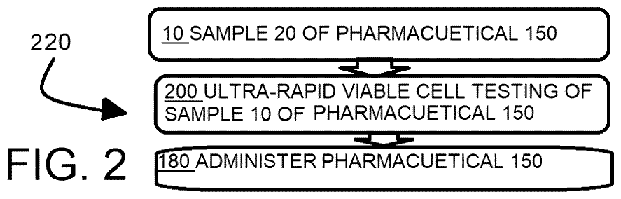

FIG. 3 is schematic flow chart of a viable cell detection method implementing LAMP amplification according to one embodiment of the present invention.

FIG. 4 schematically illustrates a kit and system for implementing the method of the present invention.

FIG. 5 is a schematic flow chart of a pharmaceutical protocol for administering a pharmaceutical to a patient according to one embodiment of the present invention.

FIG. 6 is a schematic diagram of at least a partially automated system for implementing the methods of the description.

FIG. 7 is a table showing exemplary universal conserved regions of RNaseP RNA's with relevant areas highlighted indicating common nucleotides.

FIG. 8 is an image showing a gel electrophoresis analysis of RNaseP PCR products.

FIG. 9 is an image showing a gel electrophoresis analysis of RNaseP RNA subunit RT-LAMP products.

FIG. 10 is an image showing qPCR curves for RT-qPCR experiments with dilutions of RNA obtained from revived microorganisms (Gram negative, Gram-positive, fungi).

FIGS. 11A and 11B are graphs showing the LOD data on the sensitivity of RNAse P RNA subunit detection in pre-conditioned (revived) bacterial and fungal cells.

### DETAILED DESCRIPTION

According to the description, method 200 for detecting the presence of viable cells in a sample 20 is provided. The presence of viable cells in the sample 20 is determined by detecting short-lived RNA biomarkers 40 in the sample 40 which indicate the presence of viable cells. Also described are protocols 220, detection reagents, kits 250, and systems 230, 280, and detection reagents for detecting the presence of the short-lived RNA biomarker 40.

The methods 200, protocols 220, detection reagents, kits 250, and systems 230, 280 described herein improve the capability of drug manufacturers to release drug products 150 that meet patient safety requirements, which in turn improves clinical practice. Ultra-rapid methods 200 and protocols 220, and detection reagents, kits 250, and systems 230, 280 used in the ultra-rapid methods and protocols, allow for rapid (under 1 hour) sterility testing for true pre-release sterility results for pharmaceutical products 150 with a short shelf-life, and/or those intended for immediate use are also described. Within PET pre-release sterility testing the description provides a paradigm shift in patient safety. Outside of PET, description represents a significant improvement to the drug release testing process. Implementation of the quality control (QC) processes of the description for sterile short-lived products rapid (within 1 hour) can be rapidly adopted in the pharmaceutical industry, and the same approach can be extended to other fields where rapid detection and broad group identification of viable microorganisms is of value. Further, the availability of a 1-hour 1-CFU sterility test will challenge and transform current practices well beyond short-lived products, for example by allowing conventional drug manufacturers to eliminate 14-day quarantine of their products.

The methods 200, protocols 220, detection reagents, kits 250, and systems 230, 280 described herein, including detection primers and ultra-rapid ultra-sensitive sterility testing solve an unmet need in the industry, as they meet customer's 1-hour testing requirement in context of the FDA's 1-CFU requirement and positively impact patient safety, sterility compliance, and workflow efficiency.

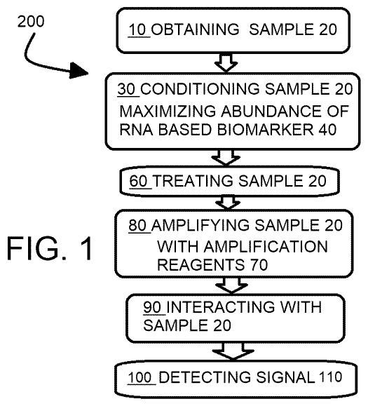

Referring now to FIG. 1, a schematic flow chart of a method 200 of sterility testing, which tests a sample 20 for viable cells, according to one embodiment of the description is provided. The viable cells and sterility to be detected by the methods 200 described herein have one or more target biomarkers 40 present on short-lived RNA molecules, i.e., short-lived RNA biomarkers 40. The short-lived RNA biomarkers 40, as described herein, are substantially abundant in viable cells and substantially non-abundant in non-viable cells. Accordingly, detection of the short-lived RNA biomarkers 40 in a threshold amount indicates the presence of viable cells, and indicates a sample is not sterile.

The present invention provides an viable cell detection method 200, such as a sterility testing method, comprising the steps of: obtaining 10 a sample 20 for detection; conditioning 30 the sample 20 for detection by facilitating an abundance of at least one short-lived RNA biomarker 40 (which may also be expressed as maximizing the abundance of the RNA based biomarker), wherein the short-lived RNA biomarker 40 is universal, namely one present in more than one kingdom of life (preferably within at least three or four kingdoms of life), abundant, namely one that is present in sufficient amounts in cells, and one that is short living, namely having a life less than 180 minutes in living cells (preferably less than 120 minutes, and more preferable less than 60 minutes); or not present in cells that are dead for 60 minutes, preferably less than 30 minutes; treating 60 the sample 20 with detection reagents 70 and contacting the short-lived RNA biomarker 40; amplifying 80 at least one nucleic acid sequence of the short-lived RNA biomarker 20 using amplification reagents 70; interacting 90 with the amplified sequences of the RNA based biomarker 40 with an detection agent to produce a readout signal 110; and detecting 100 the readout signal and determining the sterility, i.e., whether viable cells are present in the sample. It should be observed that viable cells can be preserved under cryogenic, freeze-drying, and other conditions where short-lived RNA may be found after a longer period of time.

Conditions in the analysis sample 20 are provided such that the viable cells to be detected, when present in the analysis sample 20, possess short lived RNA molecules. Optionally, conditioning reagents are provided in the conditioning step 30 and combined with the analysis sample 20 to provide at least one RNA based biomarker 40. The sample 20 is treated (step 60) to facilitate the reagents 70 contacting the short-lived RNA based biomarker 40. The treatment 60 may include combining the analysis sample 20 with RNA access components 50 (not shown) and manipulating the sample to access the short-lived RNA biomarker 40. The analysis sample 20 is then combined with detection reagents, i.e., amplification reagents 70 which interact with the manipulated sample to amplify 80 at least one nucleic acid sequence of the short-lived RNA based biomarker 20. Also included in the combined analysis sample 20 is a detection agent and optional control reagents at step 90. The detection agent which produces the read-out signal 110 which is detected at step 100 and analyzed to detect the presence of short-lived RNA biomarkers 40 in the sample 20. The presence of the detected short-lived RNA biomarkers 40 in the analysis sample 20 indicates the presence of viable cells in the analysis sample 20, or the presence of a combination of viable cells and non-viable cells. As noted above, when the detection signal of the short-lived RNA biomarkers 40 is above the threshold, which is determined by the negative control signal, the result of the method indicates the presence of viable cells, and indicates a sample is not sterile. Where the amount of the short-lived RNA biomarkers copies in a viable cell is estimated to be at least 300 copies per cell, or, preferably, at least 500-1000 copies per CFU, then in the case where the sample 20 contains less than 1000 CFU of dead cells or no cells, the read-out signal 110 will be absent or lower than the threshold amount. The presence of the signal 110 indicates the presence of viable cells. The detection of short-lived RNA biomarker nucleic acid sequences in total RNA obtained in the method workflow from viable cells above the threshold signal can also be referenced as the sensitivity of the method. The method 200 can be utilized to detect nucleic acid the short-lived RNA biomarker from as low as 1 CFU above the selected threshold signal, thus the method 200 can be properly defined as implementing a readout signal 110 which is indicative of the presence or absence of at least one viable cell within the sample 20.

Exemplary viable cells to be detected include microorganisms, more preferably pathogenic microorganisms, examples of which include gram-positive bacteria, gram-negative bacteria, and fungi. Other cells to be detected may include tumor cells, and further may include cells that underwent cell therapy. Exemplary bacteria/cubacteria include gram positive (Bacillus and other), gram negative (P. aeruginosa and other), mycobacteria (M. smegmatis and other), mycoplasma, bacteria including acrobic (E. coli and other) and anaerobic (Clostridium) bacteria). Other exemplary species include molds (aspergillus and other), yeast (Candida and other)) protista, such asamoebae, single celled eukaryotes, such as protozoa species, photosynthetic eukaryotes other than plants, chromista species, archaea/archaebacteria, including species associated with the human microbiome.

The analysis sample 20 may be a pharmaceutical 150 or radiopharmaceutical sample, such as a PET tracer, and may be a pharmaceutical 150 or radiopharmaceutical sample for delivery 180 to a patient by injection. Other embodiments include analysis samples 20 for rapid detection of viable microorganisms, such as for detection of biohazards. Other embodiments of the analysis sample 20 include cells treated with a pharmaceutical agent 150 to determine the susceptibility of the cells to the pharmaceutical agent 150, such as a drug or cancer treating agent.

The analysis sample 20 can be from 1 to 100 microliters, or larger volumes. In certain embodiments, the analysis sample 20 is from 1 to 10 microliters. In some embodiments the samples 20 are membrane filtration samples, surface swabs, or air sampling methods. In other embodiments the sample 20 is a solid tablet, drug product, or supplement. Samples 20 may also be medical devices, implantable objects, or taken therefrom. In some embodiments the sample 20 is a food or beverage product. The sample 20 may also be human specimens, such as nasopharyngeal swabs, sputum, and sterile at norm samples such as cerebral fluid, blood, and urine, and tissue bank samples. The sample 20 may be a bacterial or eukaryotic cell culture, cither native or that underwent a treatment aimed at inactivating cells or disinfecting cells.

### 10 Obtaining Sample 20

The first step for the viable cell testing method 200 of the disclosure is obtaining, at step 10 herein, a proper sample 20 of the test subject (such as a pharmaceutical 150). Obtaining a proper sample 20 includes conditions that minimize accidental contamination. As known in the art, it is vital to obtain accurate results and minimize accidental contamination being introduced during testing. For example, in sterility testing application of the method 200 of the present invention, a false positive result inevitably means that the batch or lot of subjects/products under testing will be condemned as non-sterile. For this reason, obtaining or collecting 10 the sample 20 for the method 200 described herein is preferred to follow a high level of contamination control. For example, at least equivalent to that of an aseptic filling facility. This may be associated with an ISO Class 5 cleanroom or equivalent, or an isolator may be used to provide a barrier between the laboratory environment and the test subject or product.

Compendial methods for sterility testing require that a sample be cultured using two separate media. These are usually fluid thioglycollate medium (FTM), to culture both anaerobic and some aerobic bacteria, and soybean casein digest medium (SCDM) to culture fungi and acrobic bacteria. The cultures are incubated for 14 days at 32.5° C. and 22.5° C. and then examined. Any turbidity in the culture may indicate growth and must be investigated. There are two recommended methods for carrying out the test. The first is by direct inoculation, whereby a relatively small volume of sample is removed aseptically from the sample unit and inoculated directly into a suitable volume of growth medium prior to incubation. Direct inoculation in conventional processes has some significant disadvantages. Firstly, only small volumes of product can be inoculated into the culture medium, limiting the sensitivity of the test. The present method 200 does not have these drawbacks as it is effective with small volumes, such that small volume samples 20 are perfectly acceptable, even preferred.

Returning to conventional methods, to overcome drawbacks, the recommended method for obtaining samples wherever possible is membrane filtration. In membrane filtration the sample is passed through a 0.45 μm membrane filter and the filter is then transferred to the culture medium for incubation. Membrane filtration allows the whole sample, or a composite sample, to be passed through a single filter and is therefore potentially much more sensitive than direct inoculation. Filtration also provides an opportunity to rinse away components in the sample that may cause turbidity and any growth inhibitors, such as antibiotics or preservatives, which may be present. The membrane filtration method may be carried out using a traditional ‘open’ filtration system, or by using one of the commercially available closed systems, where the sample is never exposed to the test environment, thus minimizing the opportunities for contamination and false positive results. A closed membrane filtration system may be formed with connection devices and tubing so that samples can be withdrawn aseptically from ampoules, collapsible bags and other containers without being exposed to the external environment. As discussed above, membrane filtration method may be used for obtaining samples 20 in the method 200 of the present invention.

In embodiments of the method 200 the method 200 is for ultra-rapid detection, preferably under 2 h, but other embodiments the ultra-rapid detection of viable cells is completed in under 15 min, 30 min, under 45 min, under 1 h, under 1 h 15 min, under 1 h 30 min, or under 1 h 45 min.

### 30 Conditioning Sample 20

Following obtaining the sample 20 is the step 30 of conditioning the sample 20 to facilitate an abundance (which may be described as maximizing the abundance i.e., the concentration) of the RNA biomarker 40. In the conditioning step 30, conditions are provided in the analysis sample 20 such that any viable cells in the sample 20 will have an abundance of the short-lived biomarker 40.

Primarily this conditioning 30 entails placing the sample 20 in conditioning reagents, such as media and reagents that provide conditions for the cells of interest, dormant organisms, and spores to “wake up” and metabolize for a short time, generally 20-30 minutes. This conditioning step 30 is used to stimulate cellular activity (viability) without needing to wait for replication (as required for most conventional tests), that makes the conditioning step 30 short and distinctly different from culture. This step 30 may also be referenced as a revival step 30 for RNA based biomarkers 40 to ensure the analysis sample 20 possess abundant short-lived RNA molecules 40. The revival step 30 may also include the step of removal of preservatives that can hinder the stimulation of the RNA based biomarkers 40. For some samples 20, particularly solid samples 20, may be dissolved, or a solid object is exposed to conditioning.

The conditioning step 30 provides optimal pH, temperature, and nutrients for viable cells, if present, to have normal metabolism. The procedure is preferably short (under 4, 6, 10, 20, 30, 40, 50 min, or under 1 h).

The rich media used in the revival step 30 may be a natural or synthetic cell culture media, but it does not have the same requirements of a conventional growth media that are designed for optimal growth and proliferation during culturing (over 14 days). The rich media for the revival step 30 is to revive the dormant cells rather than feed the sample for culturing and cell growth. Different types of media can be used if the test wants to change the different types of cells. Two or more conditioning reagents may also be used for conditioning 30 the cells in the analysis sample 20 where the sample 20 is split into two or more portions. The revival step 30 may include a revival period at an elevated temperature, such as 25-40° C. for a period of 5-80 minutes.

In some embodiments, different types of media are used for growing different types of cells, and in other embodiments, at least a portion of an analysis sample 30 is partitioned into at least two or more parts for conditioning 30.

In other embodiments, the cell medium is a sterile cell medium or a cell medium of known sterility. In other embodiments, two or more conditioning reagents are provided for preconditioning the cells in the analysis sample 20.

In some embodiments, the method includes a short time “conditioning” step 30, where a sample 20 is incubated with one or more conditioning reagents, e.g., rich nutritional universal medium, followed by rapidly lysing 60 or damage to the membrane of cells (if any cells are present), which is then followed by analysis of the products of the amplification steps in a contamination-free platform 280.

Methods can also be used for conditioning 30 and/or lysis 60 procedures that are optimized for a specific type of organisms—including but not limited to aerobic versus anaerobic bacteria, including but not limited to eukaryotic cells, including but not limited to fungal cells and spores.

Examples of conditioning reagents, include growth media for Cultivation of fastidious acrobic and anaerobic bacteria. Examples of suitable rich media include Brain Heart Infusion (BHI) Broth, Minimum Essential Medium, Dulbecco's Modified Eagle's Medium, Iscove's Modified Dulbecco's Medium and RPMI-1640.

Brain Heart Infusion (BHI) Broth is a media with infusion from calf brains and disodium phosphate. It exists in a few variations. Other exemplary conditioning media includes Peptic digest of sheep blood might be added to facilitate fastidious microorganisms growth, the addition of 0.1% agar can be used to lower oxygen tension to enhance the growth of anaerobic organisms. It exists in a few variations.

Additional components can be added to the media used such a buffers which support growth, neutralize additives into sample that might serve as preservatives or prevent cells metabolism in any way (such as sodium citrate, sodium benzoate, ethanol, etc. can be compensated by adding Magnesium ions, or diluting). Under some conditions, reagents lysing fungi and/or bacteria cell wall can be added simultaneously to the conditioning reagents, such as but not limited to ready lyse Lysozyme.

The conditioning or revival step 30 is capable of reviving dormant cells and spores. Data shows that RNA synthesis and protein synthesis are activated in spores within the first 15-30 min of exposure to the reviving rich medium and are essential for germination. Further data also shows permeable (active) spores with recovered metabolic activity after under 10 min of exposure to rich medium. Thus, the conditioning step 30 for reviving microorganisms ensures the selected essential target RNA 40 expression. In some embodiments, a brief nutritional stimulation (conditioning 30) of a sample 20 at physiological temperature (equal or less to 25C, 33C, 35C, 40C) for a short time (equal or less than 5 min, 10 min, 15 min, 20 min, 25 min, 30 min, 40 min, 60 min, 80 min) is provided.

### RNA Based Biomarker 40

A key aspect of the present invention is the implementation of short-lived RNA biomarkers 40 in the methodology 200 of the present invention as a universal biomarker for viable cells. It is noteworthy that not every molecule in a cell can serve as a universal biomarker of a living cell. Short-lived RNA molecules were identified as a target biomarker to detect viable cells and for rapid sterility. These short-lived RNA biomarkers 40 provide for sterility testing that has high sensitivity and universal microbial applicability. The short-lived RNA biomarkers 40 of the description are: (1) universally present in an abundance of living microorganisms, i.e., found in wide swaths of life, specifically in multiple kingdoms of life (optionally, at least three or four); (2) are short-lived and substantially not present in dead (non-viable) cells; (3) abundant in living cells to allow for high sensitivity of an assay that uses nucleic acid amplification; and (4) the target biomarker sequences are satisfactorily conserved allowing for designing degenerate universal primers for amplification.

Organisms are traditionally classified into three domains and further subdivided into one of six kingdoms of life. Organisms are placed into these categories based on similarities or common characteristics. The two main cell types are prokaryotic and eukaryotic cells. Prokaryotic organisms, such as kingdom Archaebacteria and Eubacteria, do not have a nucleus while eukaryotic organisms do. Eubacteria are what is typically referred to when discussing Bacteria, while Archaea, which were originally thought to be bacteria, have significant differences that distinguish them from the other two domains. The Eukaryotic domain includes a very diverse group of organisms, with some having characteristics of animals (protozoa), while others resemble plants (algae) or fungi (slime molds).

The RNA biomarker 40 used in the method 200 of the present invention is preferably universal, meaning they are present in wide swaths of life, specifically in in more than one kingdom of life, preferably at least three kingdoms of life, and even more preferably at least six kingdoms of life. In some embodiments, the RNA biomarkers may have regions in their sequence that are universally conserved in different species of a single phylogenetic domain, and that may be present in a plurality of species of each of the three different phylogenetic domains.

Further, the RNA biomarkers 40 are short living, within the meaning of the present application, short-lived means having a life less than 180 minutes in living cells (preferably less than 120 minutes), or not present in non-living cells after 60 minutes, preferably after 30 minutes. Not every molecule in a living cell allows for detection at low concentration over a short time. The present invention implements a type of nucleic acid testing (NAT) which identifies a portion of nucleic acids that is short-lived and abundant on a living cell, thus distinguishing from a non-viable (dead) cell. NATs differ from other tests in that interact with genetic materials (RNA or DNA) rather than antigens or antibodies. The testing methodologies are extremely sensitive, allowing detection of less than 10 copies in a reaction, but this often translates to multiple copies per analysis sample. This sensitivity can be difficult to achieve in routine testing due to inefficiencies in the extraction, transfer, reverse transcription, and amplification of the template therefore the nucleic acid target in the present invention should be highly expressed or abundant, in other words one which has a high number of molecules per cell. Abundant within the meaning of this application is a nucleic acid target present in at least 300 copies within a cell.

The method 200 of the present invention focuses on identifying living cells and a short-lived RNA biomarker 40 has been found that is substantially abundant in living cells. Substantially abundant RNA for the purpose of this application will have in the order of at least 300-1500 molecules per average prokaryotic cell. However, RNAseq data is widely available and substantially abundant RNA can be generally quantified through comparison with other molecules and with a spike in internal control in RNA seq experiments which can be performed. For further reference see, https://bionumbers.hms.harvard.edu/search.aspx.

There are several functional RNA that satisfy the requirements of universally conserved, short-lived, and abundant that can effectively form the biomarker 40 in the present invention. These include RNAzyme, IRNA, some small regulatory RNA, some transfer-messenger RNA such as SsrA tmRNA in bacteria (which serves both as a tRNA and an mRNA, releasing stalled protein biosynthesis), and some signal-recognition particles, such as 4.5S RNA in bacteria. There are a number of functional RNA that satisfy the requirements of abundant, universal and short-lived that can effectively form the biomarker 40 in the present invention. These include some mRNA encoding ribosomal subunit proteins, such as 50S and 30S ribosomal subunit proteins, such as L33, L30, L27, L35, S15. There are some coding mRNA, satisfying the desired parameters only under specific conditions, for example, mRNA coding proteins involved in amino acid biosynthesis, such as mRNA coding N-acetylglutamate synthase (ArgA) present in 28 molecules per cell when cells are in the rich medium and 1129 molecules per cell in the glucose-supplemented minimal media where the enzyme activity was highly needed. There are also a number of RNA that do not meet the requirements, most notable ribosomal RNA, such as 16s rRNA, because they are stable within dead cells.

Exemplary abundant RNA carries a household function that is always needed in a living cell. Examples include: RNAzymes (RNAse P RNA and ribosomal RNA excluded above), regulatory RNA, and tRNA are examples of abundant RNA molecules that have function beyond coding for a protein. Other examples include: mRNA coding for abundant highly conserved essential household proteins—such as for small and large subunits of ribosomal proteins, and a short lived abundant RNA molecule that performs the same basic essential function and therefore is universally present in cells of more than one kingdom of life. Examples include RNAse P RNA, as RNA is involved in maturation of the cell's machinery for translation; and tRNA is directly used for translation in all living cells. Other examples include short-lived abundant RNA molecules perform the same basic essential function and is universally present in cells of more than one kingdom of life and therefore has conserved regions in nucleic acids structure, that are associated with carrying the essential function. Particularly, the conserved regions in the nucleic acid sequences should have consensus sequences common for the large class of microorganisms and cells. For example, RNAse P RNA subunit, types and structures are described in the rfam database, https://rfam.org, with the search query “RNase p”.

RNA analysis was used to develop biomarkers 40. Ribosomal RNA (rRNA) was identified as a potential biomarker 40 as it has highly conserved universal sequences and is highly abundant, thus allowing for high sensitivity of microorganism detection. However, as rRNA can also be found in dead cells, other target biomarkers were identified to avoid accidental detection of dead cells by targeting ribosomal RNA (rRNA). RNAzyme ribonuclease P (RNAse P) 12RNA subunit was identified as a biomarker of the presence of living microorganisms' presence, as RNAse P is short-lived, highly abundant in living (viable) cells, and substantially not abundant in dead (non-viable) cells. For example, RNase E ribonuclease is present in approximately 1500 copies per cell and RNA polymerase number estimated as ˜4,500 per cell. RNAse P is expected to be found in similar concentrations, but with the specific advantage that each copy of RNAse P carries a copy of enzymatically active RNA that can be amplified and detected.

Databases confirm that the RPR subunit is universally conserved across multiple species. The Rfam database has a curated collection of 9,332 RPR sequences and their alignments; seed alignments sequences are from RNase P database, and the bigger alignments that are searches of Genbank (NCBI) using the internal search engine. It was found that bacterial RNase P is represented in at least 7,091 species; 6,324 species have class type RNase P type A, and 767 species sequences class B. Fungal nuclear RNase P RNA subunit is represented by a total of 392 species and 493 sequences. Most of the fungal sequences in the database relate to the two main Dikarya groups: (1) Ascomycota type (total 263 species and 276 sequences in classes Saccharomyceses, Sordariomycetes, Eurotiomycetes, Pneumocystidomycetes, Dothideomycetes, Pezizomycetes, Leotiomycetes, Taphrinomycetes, Xylonomycetes and other closely related types), and (2) Basidiomycota (total 83 species, 162 sequences used for the seed alignment) with the noticeable classes Agariomycetes and Tremellomycetes types among others. Other fungi phylums with closely aligned RNase PRNA subunit are Microsporidia (21 sequences and 21 species), Mucoromycota (16 sequences and 13 species), Fungiincertaesedis, Blastocladiomycota, Chytridiomycota, and Zoopagomycota. Accordingly, the satisfactory universality of the selected RPR target biomarker was confirmed in silico.

Similar assessments can be performed for other genes allowing identification of alternative biomarkers 40.

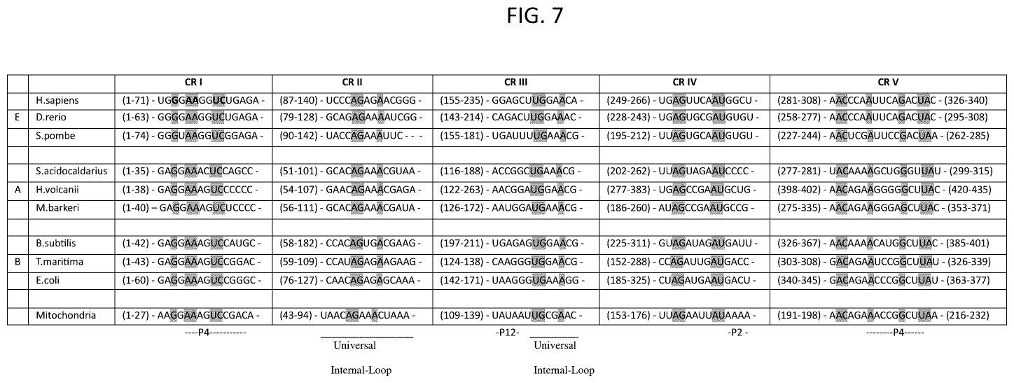

In embodiments, the target RNA biomarkers 40 are non-coding short-lived RNA molecules from the group consisting of RPR, IRNA, or small non-coding RNA. In an exemplary embodiment, the RNA subunit of Ribonuclease P (RNase P) present in living organisms was selected as the short-lived RNA biomarker for identification of living versus dead cells, as the RNase P RNA subunit (RPR) is universally (i.e., substantially abundant) in living organisms in all domains of life, including archaea, eukaryotes nuclei and in mitochondria. The RNA subunit structure is functionally conserved in diverse bacteria and is represented by two major types (A and B), and conserved across fungal species. FIG. 7 is a table showing the alignment of universally conserved regions of RNA as P RNAs, as described in RNA (1997), 3:557-560, Cambridge University Press, incorporated herein by reference in its entirety. The alignment includes three eucaryal (E), three archaeal (A), three bacterial (B), and one mitochondrial RNase P RNAs. Highlighted nucleotides indicate common nucleotides between all 10 of the RNase P RNAs shown in FIG. 7. CR sequences are indicated with bars above the alignment. Numbers between each CR are the numbers of omitted nucleotides in the alignment. Secondary structures associated with CRs are indicated below the alignment. In embodiments, the short-lived RNA biomarker 40 is a complete or partial sequence of one or more of the CR1, CR2, CR3, CR4, and CR5 conserved regions of RNase PRNA subunit.

### 60 Treating Sample 20

The method 200 of the present invention may include the step 60 of treating the sample 20 to facilitate the reagent 70 contacting the short-lived RNA biomarker 40 prior to amplifying 80 at least one nucleic acid sequence of the RNA based biomarker 40. Essentially this is manipulating the cells so that the biomarker 40 can be interacted with. To access the target biomarkers 40, the analysis sample 20 can be manipulated by chemical, biological, and physical forces to allow interaction with cell contents. Access can be accomplished by the use of RNA access components including reagents and devices which provide access to the short-lived RNA biomarkers present on the cells in the analysis sample. RNA access components can include lysis reagents release reagents, capture agents, and physical agents.

Physical methods of cell lysis for the method 200 of the present invention include mechanical disruption of cell membranes, as by repeated freezing and thawing, sonication, pressure, or filtration may also be referred to as lysis. Another physical method of cell lysis is acoustic lysis which uses ultrasonic waves to generate areas of high and low pressure which causes cavitation and in turn, cell lysis. Examples of RNA access components which are a hardware device include an electroporation cell, a bead beater, a centrifugal column, a negative pressure device, and a magnet.

Chemical methods of cell lysis for the method 200 of the present invention include chemical disruption of cell lysis which chemically deteriorates/solubilizes the proteins and lipids present within the membrane of targeted cells (this is the most commonly used lysis process in laboratories and is well known). Chemical methods of cell lysis for the method 200 of the present invention include enzymatic lysis which uses enzymes such as lysozyme or proteases to disintegrate the cell membrane 80.

Additional conditions of cell lysis for the method 200 of the present invention includes, using lysis reagents (non-ionic detergents) with additions to lysis buffer allows to minimize sorption to plastic (BSA at 1 mg/mL (over 0.01 mg/mL)) and chemicals that allow to preserve RNA (2 mM DTT (over 0.02 mM)), and RNAse inhibitors active at elevated temperature (SUPERAscIn 0.5 u/uL (over 0.005 u/uL)) with all components compatible with downstream nucleic acids detection methods without intermediate cleanups.

The method of cell lysis for the method 200 of the present invention may separate the analysis sample 20 into two or more parts for manipulation of the analysis sample 20 for access to the contents of the viable cells to be detected.

### Amplifying Sample 20 Treating 90 and Detecting 100

Following the treating 60 of the sample 20 is amplification 80 of at least one nucleic acid sequence of the RNA based biomarker 40 generally by using amplification reagents. In the following discussion it is noted that amplification can occur using a fraction of the total lysed sample

As the short-lived RNA biomarkers 40 are universally conserved, they can serve as binding sites for universal primers, referred to herein as detection reagents, detection primers, or amplification reagents which are used to detect viable cells. The amplification products of the reaction of the amplification reagents with the short-lived RNA biomarkers 40 identify the presence of viable cells. The target biomarkers nucleic sequences 40 may be present in non-viable cells in nominal amounts, e.g., from a single copy to tens of copies of the target nucleic acid sequences can be found in DNA in a non-viable cell. However, as the target nucleic acid sequences are substantially abundant in viable cells and non-abundant in non-viable cells, e.g., a 300:1, 1000:, 1500:1 ratio, the amplification products from the reaction of the amplification reagents with an analysis sample can be used to identify the presence of viable cells, even in the presence of non-viable (dead) cells and distinguished from samples with only non-viable cells or no cells. For example, an analysis sample 20 containing either viable cells, or a combination of viable cells or no cells, the amplification products can be detected in a threshold amount to identify the presence of the viable cells. In an analysis sample 20 containing non-viable cells or no cells, some amplification products may be detected but a threshold amount would not be detected. Thus, the amplification products from the reaction of the amplification reagents 70 with an analysis sample 20 can be used to detect the presence or absence of viable cells in an analysis sample 20.

Detection reagents 70, also referred to as amplification reagents, or more specifically, detection primers, for detecting the presence of a viability biomarker 40 (e.g., short-lived RNA biomarker) present on short-lived RNA molecules on viable cells is provided. When an analysis sample 20 containing the viability biomarker 40 is combined with the detection reagents 70, the reaction products, e.g., amplification products, are directly or indirectly detected in a threshold amount which indicates the presence of the viability marker from the viable cells, thus indicating the presence of viable cells in the analysis sample 20. Non-viable cells may contain the viability biomarker, or portions thereof, in a nominal amount, e.g., 1000:1 ratio of viability biomarker from viable cells vs. non-viable cells. However, the reaction products from the reaction of the detection reagent 70 with a sample 20 containing viable cells, or a combination of viable cells and non-viable cells is distinguishable from a sample 20 containing non-viable cells or no cells as a threshold amount of the detected reaction products is not detected when viable cells are not present. In one embodiment, the detection reagents 70 comprises one or more primer sets, i.e., detection primers, which target the nucleotide sequences of the viability biomarker.

One process for amplification 80 of the sample is Reverse Transcription (sometimes called transcriptase) polymerase chain reaction, RT-PCR, which is a type of PCR technique that reverse transcribes RNA to DNA and then enzymatically amplifies the DNA in vitro. RT-PCR combines the reverse transcription process with the conventional PCR process. Polymerase chain reaction (PCR) is a temperature-dependent nucleic acid amplification technique used to amplify the DNA or RNA in vitro enzymatically. PCR was developed in the mid-1980s, it is considered one of the most important tool in modern biology—molecular biology and genetics. In the RT-PCR process the sample RNA is first converted to double-stranded DNA (complementary DNA) by reverse transcriptase enzyme in the reverse transcription process. The cDNA can then be thermally broken down into two single-stranded DNA templates. In these single-stranded DNA templates, primers can anneal to their complementary sequences based on the nucleic acid hybridization principle. DNA polymerase then elongates the primer by sequentially adding the nucleotides to the 3′ end and generates a dsDNA following the principle of DNA replication. These three processes, denaturation, annealing, and elongation, are repeated in a cyclic manner regulating the reaction temperature and resulting in millions of copies of the cDNA. Novel ultra-fast PCR instrumentation, those using multiple heating zones, are an alternative to rapid PCR, demonstrating up to 30 cycles in 10-15 minutes, allowing faster amplification and detection than previously achieved. In some cases, samples can be treated with DNAses, which are designed to remove DNA prior to the RT step. This may be useful for sample where high concentrations of non-viable organisms may be expected.

One process for amplification 80 of the sample is a reverse transcription followed by an isothermal amplification such as Loop-mediated isothermal amplification (LAMP). LAMP is an isothermal nucleic acid amplification technique. In contrast to the (PCR) technology, in which the reaction is carried out with a series of alternating temperature steps or cycles, isothermal amplification is carried out at a constant temperature, and does not require a thermal cycler. This results in a faster amplification because it is not limited by the hardware changing temperatures. The use in the method 200 of the present invention may be described as a reverse transcription LAMP process (RT-LAMP) which combines LAMP with a reverse transcription step to allow the detection of RNA.

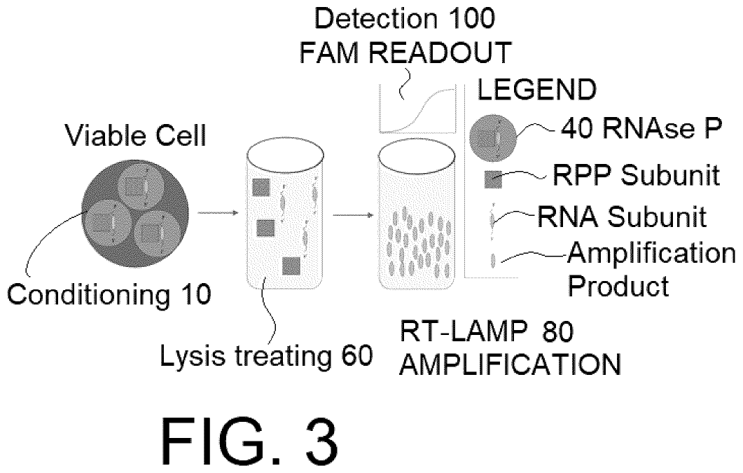

In the LAMP process of amplification 80 for the present invention, shown schematically in one example in FIG. 3, the target sequence or specifically the RNA based biomarker 40 is amplified at a constant temperature of 60-65° C. (140-149° F.) using either two or three sets of primers 70 and a polymerase with high strand displacement activity in addition to a replication activity. Typically, 4 different primers are used to amplify 6 distinct regions on the target gene, which increases specificity. An additional pair of “loop primers” can further accelerate the reaction. The amount of RNA based biomarker 40 produced in the LAMP process is generally higher than in the PCR-based amplification 80 discussed above. The LAMP process can be modified to target RNA template, rather than genomic DNA. Example: excluding B3 primer from RT-LAMP and using RNAseH active reverse transcriptase).

Primer design for primers 70 can be performed using several programs, such as PRIMEREXPLORER, MORPHOCATCHER, and NEB LAMP PRIMER DESIGN TOOL. A combination of PRIMEREXPLORER and MORPHOCATCHER may be particularly useful.

The amplification product of the LAMP process of amplification 80 can be directly detected (step 100) via photometry, measuring the turbidity caused by magnesium pyrophosphate precipitate (here the signal 110) in solution as a byproduct of amplification. This allows for simple photometric detection 100 of the signal 110 even for small volumes.

The amplification product of the LAMP process of amplification 80 can be followed in real-time either by measuring the turbidity discussed above or interacting 90 the imaging agent with the amplified sequences of the RNA based biomarker 40 to produce a readout signal 110; and detecting 100 the readout signal 110 and determining the presence or absence viable cells of the sample 20. The interacting 90 with the sample 20 may be by fluorescence using intercalating dyes such as SYTO 9. Dyes, such as SYBR green, can be used to create a visible color change as the signal 110 that can be accurately be measured or detected 100 by instrumentation.

Detection agents/imaging systems for detecting the presence of the amplification products from the reaction with the biomarker are provided. The imaging agent can comprise a colorimetric detection system, a fluorescent detection system, an electrochemical detection system, a mass spectrometry detection system, antibody detection system, or pH detection system, for example. Examples of detection agents/systems include 5′ 6-FAM™, 5′ Cy®3, 5′ SUN, 5′ Cy®5, 5′ TEX 615, 5′ TYE™ 563, 5′ TYE™ 665, 5′ HEX™, 5′ Yakima Yellow®, TET 539, YAK 549, SUN 554, HEX 555, and biotin.

Dye molecules intercalate or directly label the RNA based biomarker 40, and in turn can be correlated with the number of copies initially present. Hence, the LAMP amplification 80 can also lead to quantitative results.

Detection 100 following LAMP based amplification 80 is possible by interacting 90 with the sample 20 using manganese loaded calcein which starts fluorescing upon complexation of manganese by pyrophosphate during in vitro DNA synthesis.

Another method for detection 100 following LAMP based amplification 80 is the ability of the RNA based biomarker 40 to hybridize with complementary gold nanoparticle-bound (AuNP) single-stranded DNA (ssDNA). Thus by interacting 90 with the sample 20 the RNA based biomarker 40 hybridizing with complementary gold nanoparticle-bound (AuNP) single-stranded DNA (ssDNA) prevents the normal red to purple-blue color change that would otherwise occur during salt-induced aggregation of the gold particles, providing a signal 110 for detection 100. So, a LAMP based amplification 80 combined with amplicon detection 100 by AuNP can have advantages over other methods in terms of reduced assay time, amplicon confirmation by hybridization and use of simpler equipment (i.e., no need for a thermocycler, electrophoresis equipment or a UV trans-illuminator).

The LAMP based amplification 80 has the potential for the method 200 to be used as a simple screening assay in the field or at the point of care by clinicians. LAMP has been observed to be less sensitive (more resistant) than PCR to inhibitors in complex samples. Other advantage of the RT-LAMP based amplification that we envision, is that B3 primer can be removed to maximize the amplification that starts from RNA molecule of the biomarker, rather than from genomic DNA.

One process for amplification 80 of the sample incorporates Clustered regularly interspaced short palindromic repeats (CRISPR). Clustered Regularly Interspaced Short Palindromic Repeats (CRISPR) and CRISPR-associated (Cas) protein systems have transformed the field of genome editing and transcriptional modulation. Progress in CRISPR-Cas technology has also advanced molecular detection of diverse targets, ranging from nucleic acids to proteins. Incorporating CRISPR-Cas systems with various nucleic acid amplification strategies, PCR, isothermal, and ambient temperature enables the generation of amplified detection signals, enrichment of low-abundance molecular targets, improvements in analytical specificity and sensitivity, and development of point-of-care (POC) diagnostic techniques. These amplification and detection processes take advantage of various Cas proteins for their particular features, including RNA-guided endonuclease activity, sequence-specific recognition, multiple turnover trans-cleavage activity of Cas12 and Cas13, and unwinding and nicking ability of Cas9. Integrating a CRISPR-Cas system after nucleic acid amplification improves detection specificity due to RNA-guided recognition of specific sequences of amplicons. Incorporating CRISPR-Cas before nucleic acid amplification enables enrichment of rare and low-abundance nucleic acid targets and depletion of unwanted abundant nucleic acids. Unwinding of dsDNA to ssDNA using CRISPR-Cas9 at a moderate temperature facilitates techniques for achieving isothermal exponential amplification of nucleic acids. The integrations of CRISPR technology with nucleic acid amplification techniques result in highly sensitive and rapid detection of the RNA based biomarker 40. For further details on CRISPR incorporated amplification 80 methodologies see Feng W, Newbigging A M, Tao J, Cao Y, Peng H, Le C, Wu J, Pang B, Li J, Tyrrell D L, Zhang H, Le X C. CRISPR technology incorporating amplification strategies: molecular assays for nucleic acids, proteins, and small molecules. Chem Sci. 2021 Mar. 2; 12(13):4683-4698.

Other known amplification 80 methodologies may be utilized, including NASBA amplification that starts directly from RNA sequences. Of the above discussed processes LAMP based amplification 80 offers a cost effective efficient and effective solution.

The amplification reagents 70 are discussed above in connection with the LAMP based amplification 80. As noted above the PCR amplification employs two main reagents 70, the primers (ss DNA fragments) and a DNA polymerase.

Regarding detection 100, both real time and end-point fluorescence detection 100 can be done with fluorescent probes (Fluorescein, Cy3, TAMARA, JOE, TYE, ATTO dyes, IRDye, Rhodamine, Alexa Fluor, etc.) or with fluorescent dyes: DNA intercalating dies (Syto 9), or dyes responding to chemical changes during amplification (calcein). End-point readout or detection 100 can be done with colorimetric detection, for example, using hydroxynaphthol blue dye or phenol red pH indicator. Both real-time detection and end-point detection reagents can be combined (step 90) in the same master mixture, and both colorimetric and fluorescence-based readout or detection 100 can be used either separately or together.

One process for amplification 80 of the sample incorporates use of the biomarker 40 detecting the enzymatic activity of said biomarker. This is possible when using non-coding RNA/RNAzyme or a ribonucleoprotein complex with the non-coding RNA/RNAzyme. For example the RNAseP enzymatic activity is hydrolysis of a specific phosphodiester bond in a provided substrate (such as pre-tRNA). A fluorescent probe-quencher pair can be developed that will harness use of RNAse p bond hydrolysis to generate a signal.

Evaluation of the signals by step 100 are analyzed to determine whether the sample contain viable cells. In embodiments, the reaction products and an imaging agent, present in the sample from the reaction of the detection reagents, e.g., detection primers, with the analysis sample are evaluated and the reaction products are detectable in an amount that such that viable cells in the sample are distinguishable from a sample with non-viable cells or no cells. The presence of an detection agent, in at least a threshold amount is interpreted as an indication of the presence of the viable cells in the analysis sample, or a combination of viable cells and non-viable cells. An absence of the detection agent in at least the threshold amount indicates an absence of viable cells or no cells. As used herein, a threshold amount is determined depending on the reagents, imaging agents and systems used. However, data indicating a threshold amount of detected reaction products from a sample having viable cells is generally an amount above a negative control, and is distinguishable from the detected reaction products of a sample having non-viable cells or no cells, employing control samples where applicable.

### Pharmaceutical Treatment Protocol 200 for Pharmaceutical 150

As discussed above the culturing-based techniques are unsuitable for pharmaceutical or medical products with a short shelf-life or those intended for immediate use. The method 200 of the present invention addresses this concern. Thus the method 200 of the present invention can provide an ultra-rapid sterility testing method for a pharmaceutical product 150 including, for example, Positron Emission Tomography (PET) tracers, CAR-T cells and new cell therapy products, platelet transfusion products, gene therapies, and tissue engineered products. Another subset or grouping of the pharmaceutical products 150 for which the method 200 is particularly applicable is regenerative medicine therapy which is one of a cellular therapy, a gene therapy, and a tissue-engineered medical product. These products in the protocol 220 of the present invention may be administered 180 to patients before the standard sterility test's completion. The results obtained by execution of 200 leads to a sterility assessment and a release decision for a pharmaceutical sample.

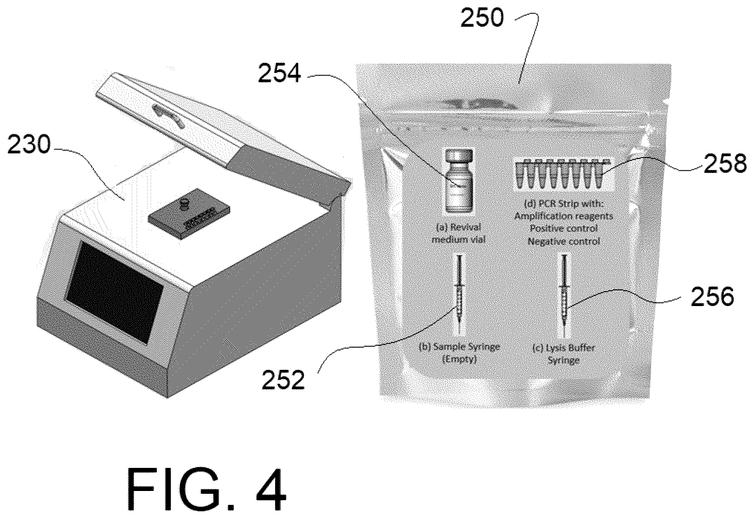

FIG. 4 schematically illustrates a system 230 and kit 250 for implementing the method 200 of the present invention suitable for point of care sterility testing method of a product 150 and for the pharmaceutical protocol 220 for administering a pharmaceutical product 150 to a patient according to the present invention. The system may include a qPCR machine 230 with a modified heating block insert and a kit 250 components for enabling all steps presented in FIG. 1: namely a) a vial 254 with concentrated sterile revival medium; b) a sterile syringe 252 for obtaining and/or transferring a test sample 20 to the vial 252 with revival medium; c) a sterile syringe 256 with pre-loaded lysis buffer; d) a nuclease-free labeled sterile PCR strip 258 with pre-loaded amplification reagents and controls

One aspect of the present invention provides a sterility testing method for a pharmaceutical product 150 comprising the steps of: obtaining 10 a sample 20 of the pharmaceutical product 150 for testing; conditioning 30 the sample 20 by facilitating an abundance or maximizing abundance of at least one universal RNA based biomarker 40; amplifying 80 at least one nucleic acid sequence of the RNA based biomarker 40 using amplification reagents 70; and detecting 100 a readout signal 110 which is indicative of the presence or absence of at least one viable cell within the sample; and determining the sterility of the sample 20 and of the pharmaceutical product 150 based upon the detected readout signal 110.

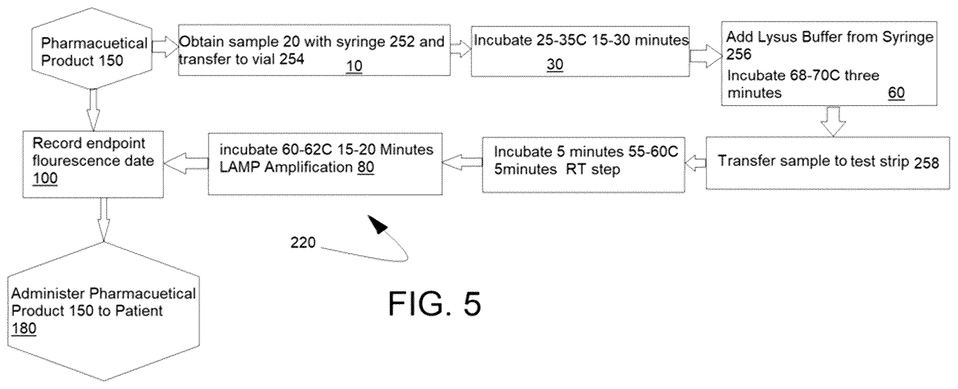

FIG. 5 is a schematic flow chart of a pharmaceutical protocol 220 for administering (at 180) a pharmaceutical 150 to a patient according to one embodiment of the present invention. The pharmaceutical protocol 220 for administering 180 a pharmaceutical 150 to a patient includes obtaining 10 a sample of a pharmaceutical 150 with syringe 252 and transferring the sample 20 to the vial 254 containing the rich medium. The vial 254 with sample 20 and rich medium is incubated for 15-30 minutes for conditioning 30 the sample 20 for testing by facilitating an abundance (or maximizing the abundance) of at least one RNA based biomarker 40. A lysis buffer is added to the vial 254 following condition 30 in a treating 60 of the sample 20. The sample 20 is transferred to the test strip 258 and a LAMP amplification 80 for amplifying at least one nucleic acid sequence of the RNA based biomarker 50 uses amplification reagents within the strip 258. The amplification 80 includes incubation for 5 minutes at 55-60 C for the RT step of the process and incubation for 15-30 minutes at 60-62C for the remaining LAMP amplification 80. The method includes detecting 100 a readout signal 110 via endpoint fluorescence which reading is indicative of the presence or absence of at least one viable cell within the sample 20. With confirmation of the sterility or safety of the sample 20 and thus the pharmaceutical product 150, the pharmaceutical product 150 is administered to the patient within three to twelve hours of obtaining the sample from the pharmaceutical. Obviously if the method fails to confirm the absence of at least one viable cell within the sample 20 the treatment 180 of the patient with the pharmaceutical 150 is postponed until a replacement pharmaceutical 150 can be found and tested.

### Kits 250

According to the description, a kit 250 for determining the presence of viable cells in an analysis sample is provided. The kit 250 may includes one or more conditioning reagents for the sample 20 conditioning of step 30. Materials in the kit 250 may be one or more sample 20 components for providing access to a biomarker of step 60. These physical materials may be a hardware device such as an electroporation cell, a bead beater, a centrifugal column, a negative pressure device and a magnet. The kit 250 may also include reagents and consumables for amplification 80 of sample 20, treating 90 with a detection agent, and detection 100. Examples of detection agents include a fluorescent label, a luminescent label, a chemiluminescent label, a radioactive label, a mass-tagged label, an optical or electrochemical label.

In certain embodiments, the materials for step 30 and step 60 are combined into a single step with a mixture of the required materials providing a one-step conditioning and lysis reagent with no intermediate steps

The kit 250 may also include one or more reaction vessels; one or more positive or negative control reagents. Detection agents can also be included in the kit 250. Examples of detection agents include a fluorescent label, a luminescent label, a chemiluminescent label, a radioactive label, a mass-tagged label, an optical or electrochemical label, as described herein.

### Systems

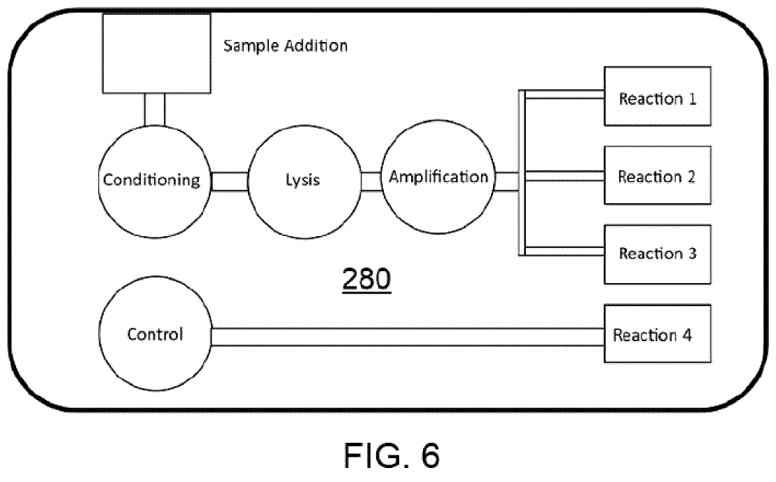

According to the description, a system, such as at least partially automated system 280 of FIG. 6, for determining presence or absence of viable cells in a sample is provided. The system 280 may include components and reagents from the kit 250 as described herein and may also include one or more reaction vessels, an area for modifying the temperature of one or more reaction vessels, an area for manipulating the cell to allow access to cell contents, and an area for biomarker detection. The area for biomarker detection may be a device that performs nucleic acid amplification using isothermal or changing temperatures using single or multiple zones.

The system 280 may be automated and be embodied into a device where the user adds a sample 20 to the cartridge and the process proceeds automatically. The device contains multiple regions, first where the user places a sample 20, the sample is transferred to the region where the conditioning step 30 takes place followed to a region where the lysis 60 takes place. This is mixed amplification 80 reagents and then transferred to detection areas 100.

In a preferred embodiment, the system 280 performs real-time measurements of the biomarker 40. In other embodiments, the system 280 is fully or partially automated, and in other embodiments, the RNA access component is a hardware device.

The method 200 may additionally incorporate the analysis of the amplified sequences for speciation of any cells found within the analysis sample 20. Although the biomarker 40 contains regions that are highly conserved, sections between them can be hypervariable between species allowing generation of a unique signature for each species.

The present invention is not limited to use with pharmaceuticals 150, but has wide application such as in particular in food or beverage production, in Tissue banks, with medical implants, within medical device manufacturing.

The invention will also have application in environmental testing such as water quality, sludge treatment, facility testing, etc.

The present invention may be used as a rapid test for susceptibility of disinfectant/treatment for microbe of interest, or for cells treated with a pharmaceutical agent to determine the susceptibility of the cells to the pharmaceutical agent, such as a drug or cancer treating agent.

A sample might include a bacterial or eukaryotic cell culture, cither native or that underwent a treatment aimed at inactivating cells or disinfecting cells

The present invention may be used in the analysis of animal specimens, such as nasopharyngeal swabs, sputum, and sterile at norm samples such as cerebral fluid, blood, and urine.

Another modification of the present invention mentioned above is the identification, following the present method and using products of amplification reaction, of specific sequences of particular pathogen of interest.

### Implementation

As the methods, kits, systems and detection agents described herein detect viable cells, a straightforward method for sterility testing is provided. The methods, kits, systems and detection agents described herein also provide for ultra-rapid ultra-sensitive sterility testing. In addition, the methods, kits, systems and detection agents described herein achieving the sensitivity required for a small number of organisms.

According to the description, a workflow for a rapid test that can detect viable cells, including active microorganisms, dormant cells, and spores with high sensitivity, but does not detect dead cells is provided. As described herein, only viable cells are detected (since dead cells will not lead to colony forming units). For sensitivity and speed of detection of the target biomolecules a three-step sample-to-answer “Ultra-Rapid Sterility Analysis” workflow, without intermediate cleanup by integrating microorganisms' reviving, lysis, isothermal amplification, and detection is provided. Referring again to FIG. 3, the 3-step workflow is shown. 1) Sample is preconditioned to ensure the presence of RNAse P. 2) Cell is lysed and ribonucleoprotein is denatured. 3) Target RNA sequence is reverse-transcribed and detected via rapid nucleic acid amplification.

To promote the RNA synthesis by the organism, the test sample is mixed with a general-purpose culture medium suitable for fastidious and non-fastidious aerobic and anaerobic microorganisms. This brief nutritional stimulation (“pre-conditioning step”) ensures the presence of RNAseP in living microorganisms and initiates revival of spores. Then, a one-step lysis suitable for single cells with no intermediate cleanup is performed, followed by amplification using degenerate primers that target conserved regions of RPR, multiplexed in six sets to detect specific groups of microorganisms. This workflow allows for universal detection of microbes within less than one hour. Because RPR is used as the target biomolecule, a single microorganism will possess an estimated 1000 copies of the sequence for amplification, allowing for the high sensitivity of the test.

Nucleic acid amplification techniques, such as RT-PCR are included as embodiments for a sterility test according to the description due to its extremely high sensitivity, its ability to amplify specific targeted sequences and detect them in a short time. One challenge with PCR-based techniques is that for low-abundance targets, the detection time even for rapid RT-PCR currently can be longer than 40-45 minutes. Accordingly, Loop-mediated isothermal amplification (LAMP) can be used according allowing for even more rapid (15-30 min), specific, and equally sensitive target sequence amplification. RT-LAMP can be performed cither using a thermocycler or a simple heat block and is known to be robust to many common DNA-polymerase inhibitors in test samples

An exemplary embodiment of a kit and system according to the description is shown in FIG. 4. As shown in FIG. 4, a qPCR machine with a customized heating block insert is provided. Kit components for performing the steps presented in FIG. 3 are also provided including: a) a vial with concentrated sterile revival medium; b) a sterile syringe for transferring a test sample to the vial with revival medium; c) a sterile syringe with pre-loaded lysis buffer; and d) a nuclease-free labeled sterile PCR strip with pre-loaded amplification reagents and controls.

An exemplary embodiment using the systems, methods, and reagents is illustrated in FIG. 6, a schematic diagram of a device illustrating the system and methods of the description. As shown in the process of FIG. 6, 1) a user injects a test sample 20 into a provided sterile nuclease-free sample area with pre-loaded concentrated sterile revival medium (conditioning reagent) 30. 2) The user loads a revival vial on the instrument and starts the protocol for pre-conditioning (30-35° C., 25° C.). The protocol pauses after the preconditioning step. 3) In a RNA access step 50, the user uses a provided sterile syringe with pre-loaded lysis buffer and adds lysis reagents to the vial with already pre-conditioned sample. The user then starts the protocol for lysis step (25° C., 60° C., 68-72° C., 4° C.). In amplification and readout steps, 4) The user transfers the lysed sample to a nuclease-free sterile PCR strip with pre-loaded frozen amplification reagents 70 using a repeating pipettor. The loading starts from test wells and ends with a positive control well. 5) User transfers the strip to the thermocycler and starts the protocol for RT-LAMP (55° C.-60° ° C. and then 60° ° C.-67° C. for defined time period). 6) The instrument provides live feedback (data) on the analysis and provides the user with answer on the presence of viable cells in the sample, preferably within an hour, with the analysis verified with positive and negative controls ensuring accuracy in the system and in data reporting.

Instructions on performing the test can be provided with the kit. Other components: racks for safe handling of a standard revival vial and a standard PCR strip, waste containers for solid waste and sharp waste, instructions on disposal of all solid waste, and engineering means to ensure sterility and radioactive safety are either provided with the kit, or standard solutions are defined in the instructions. To meet this vision, two major developments need to be completed in this project.

### EXAMPLES

### Assessment of Amplification Interference:

Different PET tracers were assessed for sources of interference with PCR due to their composition. PET tracers used in this experiment have expired and thus, were not radioactive. The effect of presence of three PET tracers (estradiol-based PET-tracer 16α-[18F]-fluoro-17β-estradiol (FES), [18F]Florbetaben (FBB) and [18F]fluorodeoxyglucose (FDG)) on the performance of two different DNA polymerases: One-step Taq Path (Applied Biosystems) and Platinum SuperFi (ThermoFisher) were tested. PET tracers were added as 10% and 30% to the PCR reaction mixture. Additionally, a concentration of 15% was used for FDG.

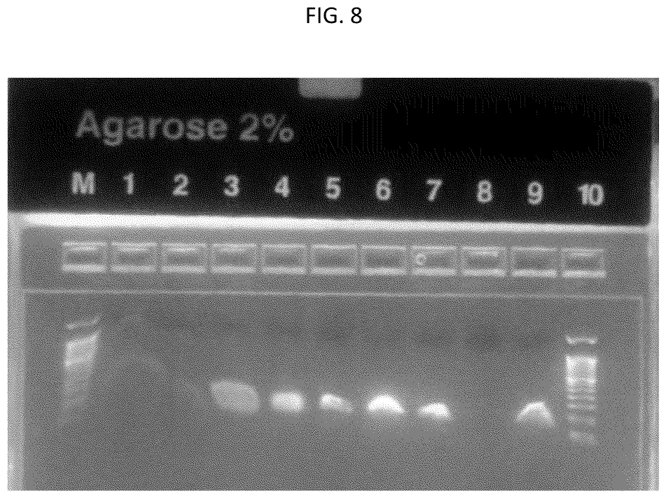

Results showed that FDG had no impact on amplification data in the qPCR mode. Then, we untangled the effect of all three PET tracers on the observed results via possible quenching a fluorescence signal in FAM channel (EvaGreen, Biotium 31090). DNA amplicons in the FDG and FBB pharmaceutical product by qPCR were seen, as well by running Gel-electrophoresis (E-Gel EX agarose Gels, 2%, Invitrogen) (FIG. 8). FIG. 8 is an image showing a gel electrophoresis analysis of RNaseP PCR products to test inhibition of DNA-polymerase by % v/v of PET-tracers (2% Agarose) Lanes M: E-Gel 50 bp DNA ladder. Sizes 50 bp-2,500 bp, reference bands at 2,500, 800, and 350 bp; 1: FES 30%; 2: FES 10%; 3: FBB 30%; 4: FBB 10%; 5: FDG 30%; 6: FDG 15%; 7: FDG 10%; 8: negative control; 9: No PET tracer added, positive control.

Only the FES samples have affected DNA-polymerase performance. We assume that inhibition happens due to the presence of up to 15% v/v ethanol in FES formulation, a known PCR inhibitor. Accordingly, the methods and reagents described herein can be applied as a sterility test to PET tracers as well as other pharmaceutical compositions, as described herein.

### Primer Design:

In an exemplary embodiment, primers at the five universally conserved regions of RNase P were developed. Pan-bacterial degenerate multiplex RT-PCR primers can be developed for such a sequence and structure for consensus types A and B. But using the structure to design RT-LAMP primers was addressed by excluding B3 primer from the set, that helps us to make reverse transcription start from B2 part of BIP primer. By excluding B3 primers we also make impossible the strand displacement by B3. Thus, accidental amplification from genomic DNA is prevented enforcing only living cell detection. RNaseH active reverse transcriptase was used to separate single-stranded cDNA from its original RNA template without denaturation step. The cDNA product of a reverse transcription with BIP primer had the tail of B1 the at the 5′ end to continue amplification reaction.

Development of a workflow that achieves 1 CFU/mL sensitivity in less than 1 hour:

After identifying a target biomarker and developing initial primer sets for molecular viability test, a rapid lysis protocol and amplification conditions were developed and optimized that allow for a highly sensitive assay.

For RT LAMP reagents from three sets of enzymes were selected: WarmStart RT-LAMP (NEB), RTx Reverse transcriptase (NEB) and BST2 polymerase (NEB) and Superscript IV_RT-LAMP system (Invitrogen) for compatibility with our three-step pre-conditioning, lysis, and amplification workflow without intermediate cleanups.

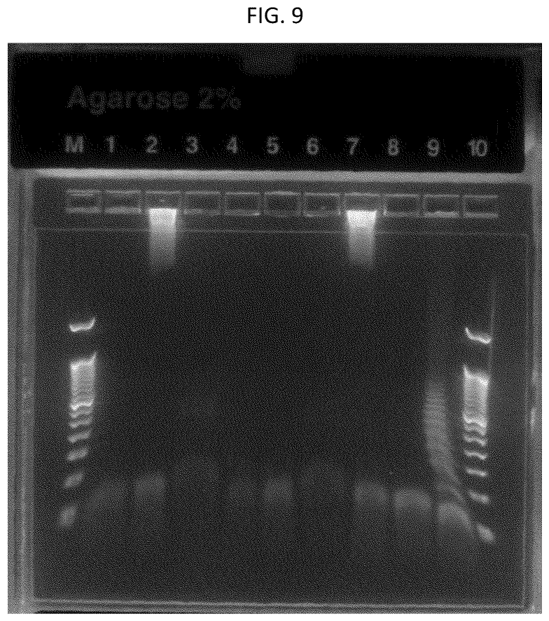

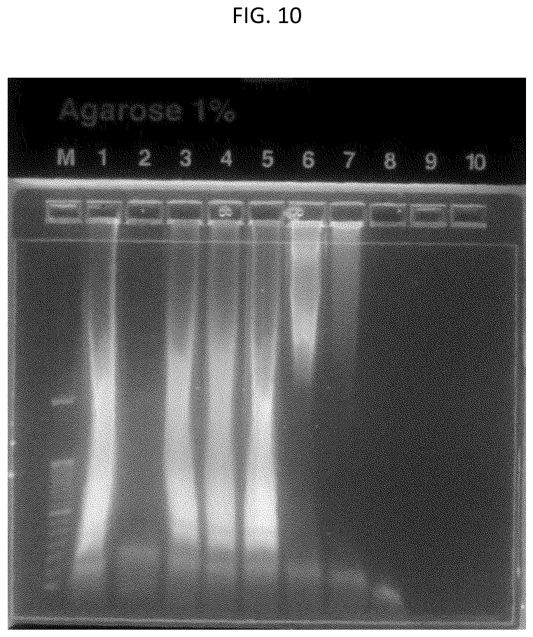

The amplification conditions were optimized, selected RNase H active reverse transcriptase to target RNA in RT-LAMP and then integrated the amplification step into the designed pipeline. FIG. 9 shows the Gram-negative bacteria RNA amplified with one universal G-RT-LAMP primer set. (2% Agarose). M: E-Gel 50 bp DNA ladder. Sizes 50 bp-2,500 bp, reference bands at 2,500, 800, and 350 bp; 1) negative control; 2) E. coli RNA); 3) Target RNA (P. aeruginosa) without added magnesium after lysis; 4,5,6) P. aeruginosa commercial quantified DNA loaded is not detected in RT-LAMP without denaturation step; 7) E. Coli RNA obtained and detected in the testing workflow; 8) E. Coli RNA (diluted 1/100), 9) P. aeruginosa RNA obtained and detected in the testing workflow; 10) E-Gel 50 bp DNA ladder. Our results showed that the signal to noise ratio was about 1000 times larger in positive samples (over 40,000 RFU) over the negative control (less than 300 RFU) within 1 hour. After that, we achieved the target sensitivity using RNA detection via RT-PCR and RT-LAMP amplification in Gram-negative viable microorganisms with LOD reaching 1 CFU (FIG. 10). FIG. 10 is a gel electrophoresis analysis of RNaseP RNA subunit RT-LAMP products. The complete workflow protocol timing was about 1 hour with the steps timed as follows: A brief nutritional stimulation (15-25 min) with Brain Heart Infusion (BHI) Broth as revival medium, followed by rapid lysis in the presence of monovalent cations, RNAse inhibitors, non-ionic detergents, and lysozyme (3-6 min) and finally rapid RT-LAMP amplification (35 min).

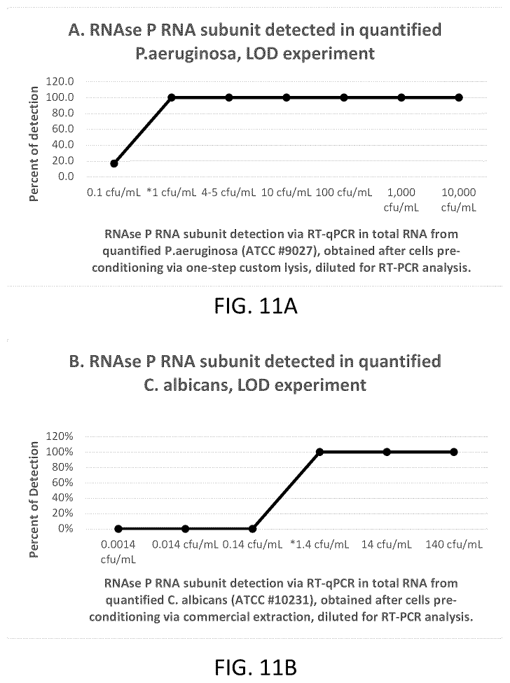

FIGS. 11A and 11B are graphs showing the LOD data on the sensitivity of RNAse P RNA subunit detection in pre-conditioned (revived) bacterial and fungal cells. FIGS. 11A and 11B show the sensitivity of detection of RNA from revived quantified bacterial stocks, obtained (A) in the developed revival/lysis/amplification testing workflow or (B) via a standard extraction using silica columns, diluted and used as a template for RNAase P detection via RT-qPCR FIG. 11A shows the LOD data for detecting RNAse P RNA subunit in P. aeruginosa ATCC #9027 mini pack frozen stock tested in the custom test workflow. Specific RNAseP RNA subunit amplicons in RNA from 1 cfu loaded per RT-PCR reaction were detected and the results shown in FIG. 11A. FIG. 11B shows the LOD data for detecting RNAse P RNA subunit C. albicans ATCC #10231 frozen stock tested in custom revival and commercial lysis using a silica column is. The specific RNAseP RNA subunit amplicons in RNA from ˜1.4 cfu/mL loaded per RT-PCR reaction were detected and the results shown in FIG. 11B. The complete workflow protocol timing was under 1 hour (for RT-LAMP) and about 2.5 h hours (for RT-PCR) with the steps timed as follows: A brief nutritional stimulation (15-25 min) with Brain Heart Infusion (BHI) Broth as revival medium, followed by rapid lysis in the presence of monovalent cations, RNAse inhibitors, non-ionic detergents, and lysozyme (3-6 min) and finally rapid RT-LAMP amplification (15-35 min) or RT-PCR amplification (1 h 15 min-1.5 h) of released target RNA in one biochemical pipeline without any intermediate cleanups.

While the foregoing written description enables one of ordinary skill to make and use what is considered presently to be the best mode thereof, those of ordinary skill will understand and appreciate the existence of variations, combinations, and equivalents of the specific embodiments, methods, and examples herein. Other embodiments will be apparent to those skilled in the art from consideration of the specification and practice of the embodiments disclosed herein. It is intended that the specification and examples be considered as exemplary only, with a true scope and spirit of the embodiment being indicated by the following claims.

1.-32. (canceled)

33. A kit for determining the presence of viable cells in an analysis sample, the kit comprising:
a) one or more conditioning reagents that facilitate conditions in the analysis sample such that the viable cells present in the analysis sample possess abundant short-lived RNA molecules;
b) one or more RNA access components for providing access to a short-lived RNAbiomarker, present in viable cells in the analysis sample, the components comprising:
i) a lysis reagent to access the short-lived RNA molecules in the analysis sample;
ii) a release reagent to access the short-lived RNA molecules in the analysis sample;
iii) a capture agent to access the short-lived RNA molecules in the analysis sample; and
iv) a physical agent to access the short-lived RNA molecules in the analysis sample; and
c) a detection agent for detecting the presence of the short-lived RNA biomarker, where the detection agent is directly or indirectly detectable to indicate the presence of short-lived RNA which is found in viable cells and not in non-viable cells.

34. The kit according to claim 33 wherein the kit further comprises one or more reaction vessels.

35. The kit according to claim 33 wherein the kit further comprises one or more positive or negative control reagents.

36. The kit according to claim 33 wherein the detection agent is a fluorescent label, a luminescent label, a chemiluminescent label, a radioactive label, a mass-tagged label, an optical or electrochemical label.

37. The kit according to claim 33 wherein the lysis reagent comprises a sterile syringe with pre-loaded lysis buffer to access the short-lived RNA molecules in the analysis sample.

38. The kit according to claim 33 wherein the detection reagents comprise materials for nucleic acid amplification.

39. The kit according to claim 33 wherein the detection reagents comprise one or more primer sets that target RPR, tRNA, or small non-coding RNA.

40. The kit according to claim 33 wherein a conditioning reagent and an RNA access component, provided as a reagent incorporated into a single vial.

41. A kit for determining the presence of viable cells in an analysis sample, the kit comprising:
one or more conditioning reagents that provide conditions in the analysis sample such that the viable cells present in the analysis sample possess abundant short-lived RNA molecules; and
one or more RT-PCR or RT-LAMP primer sets which target a short-lived RNA biomarker, where the biomarker is RNase P RNA (RPR), where primer sets anneal to a complete or partial sequence of the CR1, CR2, CR3, CR4, and CR5 conserved regions, and where the RT-PCR or RT-LAMP primer sets are directly or indirectly detectable to indicate the presence of the biomarker RNA which is found in viable cells and not in non-viable cells.

42. The kit according to claim 41 further comprising RNA access components for providing access to, short-lived RNA molecules present in viable cells in an analysis sample, the components comprising one or more of a lysis reagent, release reagent, poration consumable, or capture agent to access the short-lived RNA molecules in the analysis sample.

43. A system for determining presence or absence of viable cells in a sample comprising the kit for determining the presence of viable cells in an analysis sample according to claim 41, and further comprising:
one or more reaction vessels;
an area for modifying the temperature of one or more reaction vessels;
an area for manipulating the cell to allow access to cell contents; and
an area for biomarker detection.

44. The system according to claim 43 wherein the RNA access component is a hardware device.

45. The system according to claim 43 wherein the area for biomarker detection comprises a device that performs nucleic acid amplification using isothermal or changing temperatures using single or multiple zones.

46. The system according to claim 43 wherein the system performs real-time measurements of the biomarker.

47. The system according to claim 43 wherein the system is at least partially automated.

48. The kit according to claim 41 one or more RNA access components for providing access to a short-lived RNAbiomarker, present in viable cells in the analysis sample, the components comprising:
i) a lysis reagent to access the short-lived RNA molecules in the analysis sample;
ii) a release reagent to access the short-lived RNA molecules in the analysis sample;
iii) a capture agent to access the short-lived RNA molecules in the analysis sample; and
iv) a physical agent to access the short-lived RNA molecules in the analysis sample.

49. The kit according to claim 41 wherein the kit further comprises one or more reaction vessels.

50. The kit according to claim 41 wherein the kit further comprises one or more positive or negative control reagents.

52. The kit according to claim 33 wherein the detection agent one or more RT-PCR or RT-LAMP primer sets which target a short-lived RNA biomarker, where the biomarker is RNase P RNA (RPR), where primer sets anneal to a complete or partial sequence of the CR1, CR2, CR3, CR4, and CR5 conserved regions, and where the RT-PCR or RT-LAMP primer sets are directly or indirectly detectable to indicate the presence of the biomarker RNA which is found in viable cells and not in non-viable cells.
